(19) United States
(12) Patent Application Publication
(10) Pub. No.: US 2020/0347337 A1
(43) Pub. Date: Nov. 5, 2020

(54) METHOD OF SELECTING MICROORGANISM ISOLATES ON A HIGH-DENSITY GROWTH PLATFORM

(71) Applicant: General Automation Lab Technologies Inc., San Carlos, CA (US)

(72) Inventors: Peter Christey, San Carlos, CA (US); Alexander Hallock, San Carlos, CA (US); Maria Villancio-Wolter, San Carlos, CA (US)

(21) Appl. No.: 16/815,302

(22) Filed: Mar. 11, 2020

Related U.S. Application Data
(60) Provisional application No. 62/816,854, filed on Mar. 11, 2019.

(51) Int. Cl. C12M 1/00; C12M 1/34; C12M 1/32

(52) U.S. Cl. CPC C12M 41/06; C12M 23/12; C12M 41/26

(57) ABSTRACT

Provided is a method of using a device including a plurality of microscale experimental units. At least one cell and an indicator capable of producing an optical signal are loaded in each of the plurality of experimental units. The device with the loaded contents is incubated to grow a plurality of cells. At least one optical property of the contents of each of the plurality of experimental units is measured at multiple time points during the incubation to thereby obtain a time-dependent profile. The time-dependent profile for the plurality of experimental units are analyzed, and the analysis result may be used to select/transfer contents from certain experimental unit(s) for further cultivation and/or assay, or to determine whether certain experimental units contain any biological entity of interest.

### CROSS-REFERENCE TO RELATED APPLICATION

This application claims priority to U.S. Provisional Patent Application No. 62/816,854, filed Mar. 11, 2019, the disclosure of which is incorporated by reference herein in its entirety.

### BACKGROUND

In the study of cultured cells, one often need to measure the rate at which those cells divide or proliferate. Measuring the rate of growth (or cell division) can provide valuable information about basic health and cell maintenance, as well as the responses to particular drugs.

One common method of assessing cell proliferation looks at the entire DNA content in cells, either over time or as an endpoint assay. Another method of measuring proliferation is based on metabolism of cells, for example, by using tetrazolium salts such as MTT, MTS or XTT (the salts are reduced by metabolically active cells to a colored formazan, which is then detected using a spectrophotometer).

Resazurin is a blue dye and a common redox indicator used in cell-proliferation or viability assays. It is weakly fluorescent until irreversibly reduced to the pink colored and highly red fluorescent resorufin when around metabolically active cells.

### SUMMARY

In one aspect, the present disclosure provides a method of using a device comprising a plurality of microscale experimental units. The method comprises: in each of the plurality of experimental units, providing at least one cell and an indicator capable of producing an optical signal; incubating the device at predetermined conditions for a duration of time to grow a plurality of cells from the at least one cell in each of the plurality of experimental units; measuring at least one optical property of the contents of each of the plurality of experimental units at multiple time points during the incubation, thereby obtaining a time-dependent profile of the optical property for each of the plurality of experimental units; and analyzing the time-dependent profiles of the at least one optical property for the plurality of experimental units. The method can further comprise: based on the analysis of the time-dependent profiles of the at least one optical property for the plurality of experimental units, determining the presence or absence of a biological entity of interest, such as a eukaryotic cell or bacterial cell, in at least one of the plurality of experimental units.

In some embodiments, the analysis of the time-dependent profiles of the at least one optical property for the plurality of experimental units comprises a comparison across the time-dependent profiles of the at least one optical property for the plurality of experimental units. In certain embodiments, the method further comprises: if the time-dependent profiles of the at least one optical property of two or more of the plurality of experimental units are determined to be sufficiently similar, transferring some of the plurality of cells from only one of the two or more experimental units to a target location, or from a smaller subset of the two or more experimental units each to a respective target location.

In some embodiments, the analysis of the time-dependent profiles of the at least one optical property for the plurality of experimental units comprises identifying one or more features of the time-dependent profiles. The one or more features can comprise one or more of: the intensity of the optical property at particular time points, the ratio of the intensity of the at least one optical property at different wavelengths, and the rate of change of the at least one optical property with time.

In some embodiments, the method further comprises: based on the analysis of the time-dependent profiles of the at least one optical property for the plurality of experimental units, transferring some of the plurality of cells from at least one of the plurality of experimental units to at least one target location.

In some embodiments, the method further comprises: if the time-dependent profiles of the at least one optical property of two of the plurality of experimental units are determined to be sufficiently dissimilar, transferring some of the plurality of cells from each of the two experimental units to a respective target location.

In some embodiments, the at least one optical property of the indicator comprises fluorescence. In some embodiments, the indicator is capable of emitting fluorescence signals of different wavelength at different redox states. In some embodiments, the indicator is resazurin. In some embodiments, the indicator is pH sensitive.

In some embodiments, providing the at least one cell comprises loading into each of the plurality of experimental units only one cell.

In some embodiments, the device is a microfabricated device having a top surface defining an array of microwells as experimental units. Each microwell of the plurality of microwells can have a diameter of about 25 μm to about 500 μm. The surface density of the plurality of microwells can be at least 750 microwells per cm².

In another aspect, the present disclosure provides a method of using a device comprising a plurality of microscale experimental units. The method comprises: in each of a plurality of experimental units, providing at least one cell and an indicator capable of producing an optical signal; incubating the device at predetermined conditions for a duration of time to grow a plurality of cells from the at least one cell in each of the plurality of experimental units; measuring at least one optical property of the contents of each of the plurality of experimental units during or after the incubation; analyzing the measured at least one optical property for each of the plurality of experimental units; and based on the analysis, selecting some of the plurality of cells in one or more of the plurality of experimental units.

In some embodiments, measuring at least one optical property comprises measuring the at least one optical property of the contents of each of the plurality of experimental units at multiple time points during the incubation.

In some embodiments, analyzing the measured at least one optical property for each of the plurality of experimental units comprises a comparison among the at least one optical property of each of the plurality of experimental units measured at a same time point.

In some embodiments, analyzing the measured at least one optical property for each of the plurality of experimental units comprises a comparison among the at least one optical property of each of the plurality of experimental units measured at multiple time points.

In some embodiments, the method further comprises: if the measured at least one optical property of two or more of the plurality of experimental units are determined to meet a similarity threshold, transferring some of the plurality of cells from only one of the two or more experimental units to a target location, or from a smaller subset of the two or more experimental units each to a respective target location.

In some embodiments, analyzing the measured at least one optical property for each of the plurality of experimental units comprises calculating the ratio of the intensity of the at least one optical property measured at a same time at different wavelengths.

### BRIEF DESCRIPTION OF THE DRAWINGS

FIG. 1 is a perspective view illustrating a microfabricated device or chip in accordance with some embodiments of the present disclosure.

FIGS. 2A-2C are top, side, and end views, respectively, illustrating dimensions of microfabricated device or chip in accordance with some embodiments of the present disclosure.

FIGS. 3A and 3B are exploded and top views, respectively, illustrating a microfabricated device or chip in accordance with some embodiments of the present disclosure.

FIG. 4 shows a testing result using resazurin as indicator on a microfabricated chip on two different bacterial strains in accordance with some embodiments of the present disclosure.

FIG. 5 includes a snapshot of wells on a microfabricated chip and a time-dependent fluorescence signals of resazurin for the wells, in accordance with some embodiments of the present disclosure.

FIG. 6 shows time-course behavior of well contents on a microfabricated chip with resazurin as indicator, at two fluorescence channels (green/red) over time, in accordance with some embodiments of the present disclosure.

### DETAILED DESCRIPTION

The present disclosure relates generally to systems and methods for isolation, culturing, sampling, and/or screening of biological entities. One object of the disclosed subject matter is to provide a method to track, analyze and/or screen cell isolates based on cell growth, metabolic activity, and/or viability on a high density cell cultivation platform.

The methodology of the present disclosure is based on a highly partitioned system or platform which comprises a high density array or arrays of microscale experimental units, where each microscale experimental unit can accommodate one or more cells and provide an environment independent and separate from other microscale experimental units for cell growth and proliferation.

In some embodiments, the high density cell cultivation platform can be a microfabricated device (or a “chip”) for receiving a sample comprising at least one biological entity (e.g., at least one cell). The term “biological entity” may include, but is not limited to, an organism, a cell, a cell component, a cell product, and a virus, and the term “species” may be used to describe a unit of classification, including, but not limited to, an operational taxonomic unit (OTU), a genotype, a phylotype, a phenotype, an ecotype, a history, a behavior or interaction, a product, a variant, and an evolutionarily significant unit.

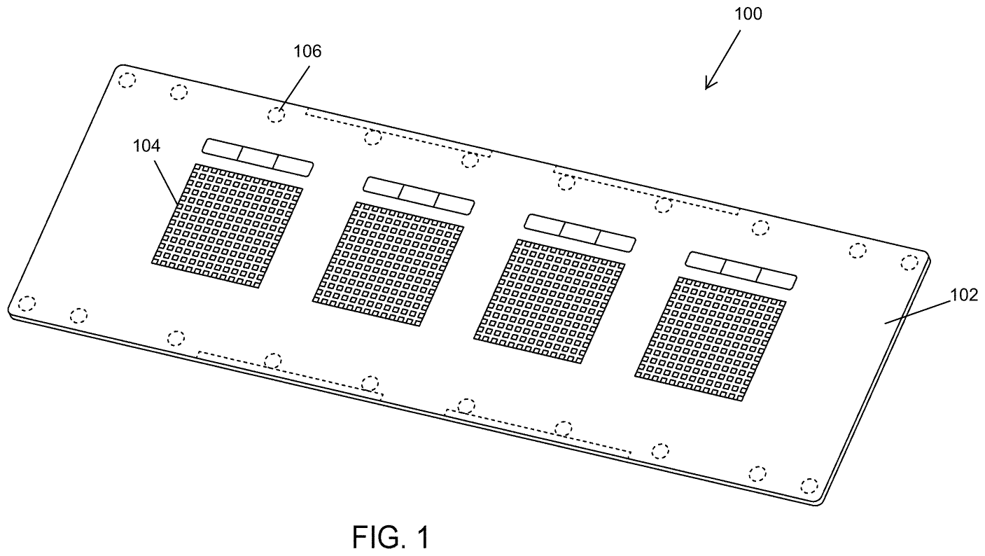

As used herein, a microfabricated device or chip may define a high density array of microwells (or experimental units). For example, a microfabricated chip comprising a “high density” of microwells may include about 150 microwells per cm² to about 160,000 microwells or more per cm² (for example, at least 150 microwells per cm², at least 250 microwells per cm², at least 400 microwells per cm², at least 500 microwells per cm², at least 750 microwells per cm², at least 1,000 microwells per cm², at least 2,500 microwells per cm², at least 5,000 microwells per cm², at least 7,500 microwells per cm², at least 10,000 microwells per cm², at least 50,000 microwells per cm², at least 100,000 microwells per cm², or at least 160,000 microwells per cm²). A substrate of a microfabricated chip may include about or more than 10,000,000 microwells or locations. For example, an array of microwells may include at least 96 locations, at least 1,000 locations, at least 5,000 locations, at least 10,000 locations, at least 50,000 locations, at least 100,000 locations, at least 500,000 locations, at least 1,000,000 locations, at least 5,000,000 locations, or at least 10,000,000 locations. The arrays of microwells may form grid patterns, and be grouped into separate areas or sections. The dimensions of a microwell may range from nanoscopic (e.g., a diameter from about 1 to about 100 nanometers) to microscopic. For example, each microwell may have a diameter of about 1 μm to about 800 μm, a diameter of about 25 μm to about 500 μm, or a diameter of about 30 μm to about 100 μm. A microwell may have a diameter of about or less than 1 μm, about or less than 5 μm, about or less than 10 μm, about or less than 25 μm, about or less than 50 μm, about or less than 100 μm, about or less than 200 μm, about or less than 300 μm, about or less than 400 μm, about or less than 500 μm, about or less than 600 μm, about or less than 700 μm, or about or less than 800 μm. In exemplary embodiments, the diameter of the microwells can be about 100 μm or smaller, or 50 μm or smaller. A microwell may have a depth of about 25 μm to about 100 μm, e.g., about 1 μm, about 5 μm, about 10 μm, about 25 μm, about 50 μm, about 100 μm. It can also have greater depth, e.g., about 200 μm, about 300 μm, about 400 μm, about 500 μm. The microfabricated chip can have two major surfaces: a top surface and a bottom surface, where the microwells have openings at the top surface. Each microwell of the microwells may have an opening or cross section having any shape, e.g., round, hexagonal, square, or other shapes. Each microwell may include sidewalls. For microwells that are not round in their openings or cross sections, the diameter of the microwells described herein refer to the effective diameter of a circular shape having an equivalent area. For example, for a square shaped microwell having side lengths of 10×10 microns, a circle having an equivalent area (100 square microns) has a diameter of 11.3 microns. Each microwell may include a sidewall or sidewalls. The sidewalls may have a cross-sectional profile that is straight, oblique, and/or curved. Each microwell includes a bottom which can be flat, round, or of other shapes. The microfabricated chip (with the microwells thereon) may be manufactured from a polymer, e.g., a cyclic olefin polymer, via precision injection molding or some other process such as embossing. Other material of construction is also available, such as silicon and glass. The chip may have a substantially planar major surface. FIG. 1 shows a schematic depiction of a microfabricated chip, whose edges are generally parallel to the directions of the rows and the columns of the microwells on the chip.

In some embodiments, the high density cell cultivation platform can be droplet based, e.g., instead of array(s) of wells as experimental units on a microfabricated chip, a population of discrete droplets can be used as experimental units to retain cell, media and other components for cell cultivation. Droplet generation methods, especially when combined with cell-sorter-on-a-chip type instrumentation, may be used to grow and screen microbes from a complex environmental sample. Droplets may be produced at several hundred Hz, meaning millions of drops can be produced in a few hours. A simple chip-based device may be used to generate droplets and the droplets may be engineered to contain a single cell. A system for generating droplets containing cell suspensions may contain one or small numbers of cells. The droplets can be emulsions, double emulsion, hydrogel, bubbles and complex particles, etc. For example, aqueous drops may be suspended in a nonmiscible liquid keeping them apart from each other and from touching or contaminating any surfaces. The volume of a droplet can be somewhere between 10 fl and 1 μL, and highly monodisperse droplets can be made from a few nanometers up to 500 μm in diameter.

A droplet-based microfluidic system may be used to generate, manipulate, and/or incubate small droplets. Cell survival and proliferation can be similar to control experiments in bulk solution. Fluorescence screening of droplets may be done on-chip and at a rate of, for example, 500 drops per second. Droplets may be merged to create a new droplet or a reagent added to a droplet. Droplets can be passed in a microchannel in a single file and interrogated by a spectroscopic method, e.g., using a fluorescence detector to detect fluorescence emitted from the droplets, and those droplets that are determined to meet certain criteria (e.g., emitting fluorescence at certain wavelength) can be selected via diversion into a branched channel from which the droplet can be pooled or harvested. The diversion or switching of flow can be accomplished by valves, pump, applying an external electric field, etc.

The high density microwells on the microfabricated chip can be used to conduct various experiments, such as growth or cultivation or screening of various species of bacteria and other microorganisms (or microbes) such as aerobic, anaerobic, and/or facultative aerobic microorganisms. The microwells may be used to conduct experiments with eukaryotic cells such as mammalian cells. Also, the microwells can be used to conduct various genomic or proteomic experiments, and may contain cell products or components, or other chemical or biological substances or entities, such as a cell surface (e.g., a cell membrane or wall), a metabolite, a vitamin, a hormone, a neurotransmitter, an antibody, an amino acid, an enzyme, a protein, a saccharide, ATP, a lipid, a nucleoside, a nucleotide, a nucleic acid (e.g., DNA or RNA), a chemical, e.g., a dye, enzyme substrate, etc.

A cell may be Archaea, Bacteria, or Eukaryota (e.g., fungi). For example, a cell may be a microorganism, such as an aerobic, anaerobic, or facultative aerobic microorganisms. A virus may be a bacteriophage. Other cell components/products may include, but are not limited to, proteins, amino acids, enzymes, saccharides, adenosine triphosphate (ATP), lipids, nucleic acids (e.g., DNA and RNA), nucleosides, nucleotides, cell membranes/walls, flagella, fimbriae, organelles, metabolites, vitamins, hormones, neurotransmitters, and antibodies.

For the cultivation of cells, a nutrient is often provided. A nutrient may be defined (e.g., a chemically defined or synthetic medium) or undefined (e.g., a basal or complex medium). A nutrient may include or be a component of a laboratory-formulated and/or a commercially manufactured medium (e.g., a mix of two or more chemicals). A nutrient may include or be a component of a liquid nutrient medium (i.e., a nutrient broth), such as a marine broth, a lysogeny broth (e.g., Luria broth), etc. A nutrient may include or be a component of a liquid medium mixed with agar to form a solid medium and/or a commercially available manufactured agar plate, such as blood agar.

A nutrient may include or be a component of selective media. For example, selective media may be used for the growth of only certain biological entities or only biological entities with certain properties (e.g., antibiotic resistance or synthesis of a certain metabolite). A nutrient may include or be a component of differential media to distinguish one type of biological entity from another type of biological entity or other types of biological entities by using biochemical characteristics in the presence of specific indicator (e.g., neutral red, phenol red, eosin y, or methylene blue).

A nutrient may include or be a component of an extract of or media derived from a natural environment. For example, a nutrient may be derived from an environment natural to a particular type of biological entity, a different environment, or a plurality of environments. The environment may include, but is not limited to, one or more of a biological tissue (e.g., connective, muscle, nervous, epithelial, plant epidermis, vascular, ground, etc.), a biological fluid or other biological product (e.g., amniotic fluid, bile, blood, cerebrospinal fluid, cerumen, exudate, fecal matter, gastric fluid, interstitial fluid, intracellular fluid, lymphatic fluid, milk, mucus, rumen content, saliva, sebum, semen, sweat, urine, vaginal secretion, vomit, etc.), a microbial suspension, air (including, e.g., different gas contents), supercritical carbon dioxide, soil (including, e.g., minerals, organic matter, gases, liquids, organisms, etc.), sediment (e.g., agricultural, marine, etc.), living organic matter (e.g., plants, insects, other small organisms and microorganisms), dead organic matter, forage (e.g., grasses, legumes, silage, crop residue, etc.), a mineral, oil or oil products (e.g., animal, vegetable, petrochemical), water (e.g., naturally-sourced freshwater, drinking water, seawater, etc.), and/or sewage (e.g., sanitary, commercial, industrial, and/or agricultural wastewater and surface runoff).

FIG. 1 is a perspective view illustrating a microfabricated device or chip in accordance with some embodiments. Chip 100 includes a substrate shaped in a microscope slide format with injection-molded features on top surface 102. The features include four separate microwell arrays (or microarrays) 104 as well as ejector marks 106. The microwells in each microarray are arranged in a grid pattern with well-free margins around the edges of chip 100 and between microarrays 104.

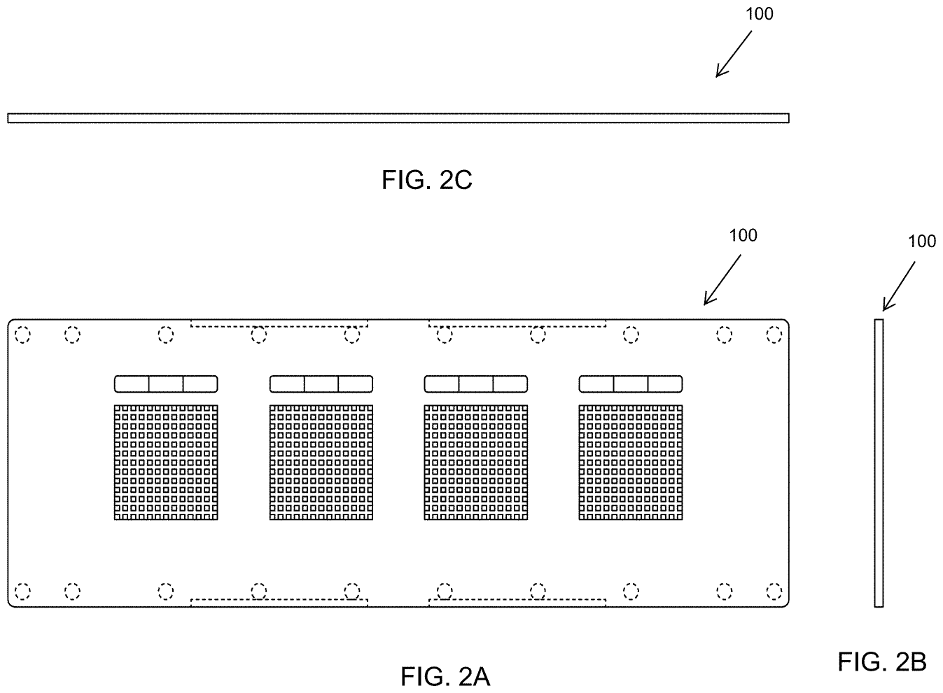

FIGS. 2A-2C are top, side, and end views, respectively, illustrating dimensions of chip 100 in accordance with some embodiments. In FIG. 2A, the top of chip 100 is approximately 25.5 mm by 75.5 mm. In FIG. 2B, the end of chip 100 is approximately 25.5 mm by 0.8 mm. In FIG. 2C, the side of chip 100 is approximately 75.5 mm by 0.8 mm.

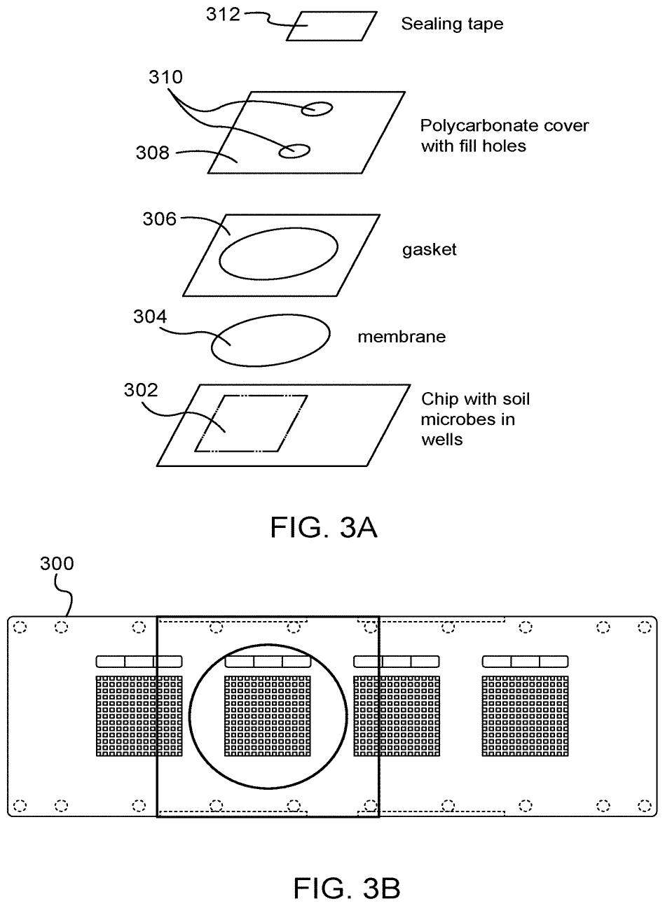

After a sample is loaded on a microfabricated device, a membrane may be applied to at least a portion of a microfabricated device. FIG. 3A is an exploded diagram of the microfabricated device 300 shown from a top view in FIG. 3B in accordance with some embodiments. Device 300 includes a chip with an array of wells 302 holding, for example, soil microbes. A membrane 304 is placed on top of the array of wells 302. A gasket 306 is placed on top of the membrane 304. A cover 308 with fill holes 310 is placed on top of the gasket 306. Finally, sealing tape 312 is applied to the cover 308.

A membrane may cover at least a portion of a microfabricated device including one or more experimental units, wells, or microwells. For example, after a sample is loaded on a microfabricated device, at least one membrane may be applied to at least one microwell of a high density array of microwells. A plurality of membranes may be applied to a plurality of portions of a microfabricated device. For example, separate membranes may be applied to separate subsections of a high density array of microwells.

A membrane may be connected, attached, partially attached, affixed, sealed, and/or partially sealed to a microfabricated device to retain at least one biological entity in the at least one microwell of the high density array of microwells. For example, a membrane may be reversibly affixed to a microfabricated device using lamination. A membrane may be punctured, peeled back, detached, partially detached, removed, and/or partially removed to access at least one biological entity in the at least one microwell of the high density array of microwells.

A portion of the population of cells in at least one experimental unit, well, or microwell may attach to a membrane (via, e.g., adsorption). If so, the population of cells in at least one experimental unit, well, or microwell may be sampled by peeling back the membrane such that the portion of the population of cells in the at least one experimental unit, well, or microwell remains attached to the membrane.

A membrane may be impermeable, semi-permeable, selectively permeable, differentially permeable, and/or partially permeable to allow diffusion of at least one nutrient into the at least one microwell of a high density array of microwells. For example, a membrane may include a natural material and/or a synthetic material. A membrane may include a hydrogel layer and/or filter paper. In some embodiments, a membrane is selected with a pore size small enough to retain at least some or all of the cells in a microwell. For mammalian cells, the pore size may be a few microns and still retain the cells. However, in some embodiments, the pore size may be less than or equal to about 0.2 μm, such as 0.1 μm. An impermeable membrane has a pore size approaching zero. It is understood that the membrane may have a complex structure that may or may not have defined pore sizes

In one aspect, the present disclosure provides methods of operating a microfabricated device having a top surface defining an array of microwells, as those microfabricated devices described herein. The methods can be used for screening or determining at least one biological entity of interest in a sample, or for cultivating, analyzing, and processing isolates of microorganisms.

Using conventional methods, such as resazurin for the high density cell cultivation platforms would be difficult to distinguish the contents of one experimental unit from another if many wells (or droplets, etc.) display similar fluorescent properties. If it can be determined these experimental units all contain the same species of cells, downstream analysis can be done by picking/transferring some cells from these experimental units. The methods disclosed herein can be used to accomplish this purpose, and others.

In some embodiments of the methods, into each of a plurality of experimental units of a device, the following materials are loaded: (a) at least one cell from a sample; (b) optionally an amount of a nutrient; (c) an indicator capable of producing an optical signal. If the device is a microfabricated device and the experimental units are microwells on the microfabricated device, a cover film may be applied to the microfabricated device to retain the at least one cell in each of the plurality of microwells. The device with the loaded materials is then incubated at predetermined conditions for a duration of time to grow a plurality of cells from the at least one cell in each of the plurality of experimental units. One or more optical properties of the contents of each of the plurality of experimental units (or simply, the optical property of the experimental units) can be measured at one or more time points during and/or after the incubation. If multiple time points measurements are taken (for example, any number of measurements from 2 to 1000, or from 2 to 100, or from 2 to 10, e.g., 2, 3, 4, 5, 6, 7, 8, 9, 10), a time-dependent profile of the at least one optical property for each of the plurality of experimental units can be constructed from these measurements. In some embodiments, at least 3 time points measurements are taken. In some embodiments, at least 4 time points measurements are taken. In some embodiments, at least 5 time points measurements are taken. In some embodiments, at least 10 time points measurements are taken. In some embodiments, at least 20 time points measurements are taken. In some embodiments, at least 30 time points measurements are taken. In some embodiments, at least 50 time points measurements are taken. In some embodiments, at least 100 time points measurements are taken. In some embodiments, at least 200 time points measurements are taken. In some embodiments, at least 500 time points measurements are taken. In some embodiments, at least 1000 time points measurements are taken. The time intervals for taking the multiple time points measurements can be in regular, for example, the optical property (or properties) of the experimental units can be measured every 10 minutes, every 20 minutes, every 30 minutes, every hour, every 2 hours, every 3 hours, every 4 hours, etc., or the time intervals can be irregular or dynamically changed during the course of the observation and selected based on the results of the previous measurements.

The single-point measurements of the at least one optical property, or time-dependent profiles of the at least one optical property for the plurality of experimental units can be analyzed. The analysis can provide a wealth of information and different subsequent actions can be taken. In certain embodiments, the presence or absence of a biological entity of interest in at least one experimental unit in the plurality of experimental units can be determined based on the analysis (e.g., if the profile of the biological entity of interest is previously known). Additionally or alternatively, based on the analysis, a decision can be made as to which experimental unit(s) in the plurality of experimental units will be selected, from which a portion of the cells can be picked and transferred to a new location or growth environment for downstream work and analysis. For example, based on the analysis, the cell isolates in the experimental units (after incubation) can be classified into different groups, and cell isolates in select experimental units (not all experimental units) from each group can be picked/transferred for downstream work.

In some embodiments, if a biological entity of interest is determined to be present in at least one experimental unit, some of the plurality of cells from the at least one experimental unit can be transferred to a target location, e.g., to a cell culture media for further growth/cultivation, or for further identification and analysis (e.g., DNA sequencing). As used in connection with the number of cells herein, the word “some” in this application means one or more.

In some embodiments, the analysis of the time-dependent profiles of the optical property for the plurality of experimental units comprises a comparison across the time-dependent profiles of the optical property for the plurality of experimental units. For example, in some embodiments, if the time-dependent profiles of the optical property of two or more of the plurality of experimental units are determined to be sufficiently similar, some of the plurality of cells can be transferred from only one of the two or more experimental units to a target location, or from a smaller subset of the two or more experimental units each to a respective target location. In this way, a user of the microfabricated chip can recover cell isolates with better diversity for downstream analysis. The similarity (or dissimilarity) of two time-dependent profiles or curves can be based on predetermined criteria, such as the normalized mean square distance on the signal strength coordinate between the two curves, normalized areas under the two curves, or by a Kolmogorov-Smirnov test. The threshold for similarity test can vary depending on the exact application. For example, it can depend on how many target locations or spots are available or desired for transferring cell isolates.

In some embodiments, the analysis of the time-dependent profiles of the at least one optical property for the plurality of experimental units comprises identifying one or more features of the time-dependent profiles. Such features can include: the characteristics of the at least one optical property at particular time points, the intensity of the at least one optical property at particular time points, the ratio of the intensity of the at least one optical property at different wavelengths, the rate of change of the at least one optical property with time, general trend of the at least one optical property between certain time window, number of inflection points of the time-dependent profile, etc. These extracted features can be used to distinguish between the different time-dependent profiles, and can be considered as a preparation step before running a comparison between different time-dependent profiles.

In some embodiments, if the time-dependent profiles of the optical property of two of the plurality of experimental units are determined to be dissimilar, some of the plurality of cells from each of the two experimental units can be transferred to a respective target location, e.g., to a cell culture media for further growth/cultivation, or for further identification and analysis (e.g., DNA sequencing). Given limited number of target locations, one may choose those experimental units on the high density cell cultivation device having the most diverse observed optical properties or time-course profiles of the optical properties to transfer to respective target locations in order to improve the diversity of the cell isolates harvest.

One or more optical properties of each experimental unit can be monitored individually using image capturing and analysis hardware and software. The optical property can be fluorescence, phosphorescence, other luminescence properties, optical density, light scattering properties, Raman emission, or simply the color of the indicator (i.e., colorimetry). Two or more the optical properties of a same indicator (optical agent), or two of more indicators having different optical properties can be used in combination to yield a richer data set for analysis.

The optical property of each of the experimental units can be measured simultaneously (or within very short time intervals, e.g., within seconds) at one or more particular time points during the incubation. If the measurements are taken multiple times (preferably with good frequency), a time-course profile of the change of the optical property can be obtained.

The indicator can be a compound or substance capable of emitting fluorescence signals of different wavelengths at different redox states. For example, the indicator can be resazurin. In other examples, the indicator can be a pH indicator that is sensitive to the pH of the surrounding medium. In further examples, optical density of the experimental units can be used as the basis of the analysis and determination as to what subsequent action to take.

In some embodiments, the at least one biological entity of interest comprises a eukaryotic cell or bacteria. In some embodiments, the sample comprise a plurality of microbial cells of different species or genera.

In some embodiments, loading is done in a manner that into each of the plurality of experimental units, on average one cell is loaded. Preferably, each of the experimental units is loaded with one cell. Or, a subset of the experimental units each is loaded with only one cell (and other experimental units are not loaded with any cells). In this way, the cell isolates growing in each experimental unit will be guaranteed to be a single species rather than a mixture of different species.

In some embodiments, each of the experimental units has a diameter of about 25 μm to about 500 μm. In some embodiments, the surface density of the array of experimental units is at least 750 microwells per cm². In some embodiments, the spacing between two neighboring experimental units in the array of the microwells is less than 500 μm (center to center).

### Example 1

A feature of the resazurin/resorufin system is that resorufin can be further reduced into dihydroresorufin which is, itself, nonfluorescent. This can often be a problem because it represents a loss of signal in the system. However, if time courses of the fluorescence are followed, different microbes can be distinguished.

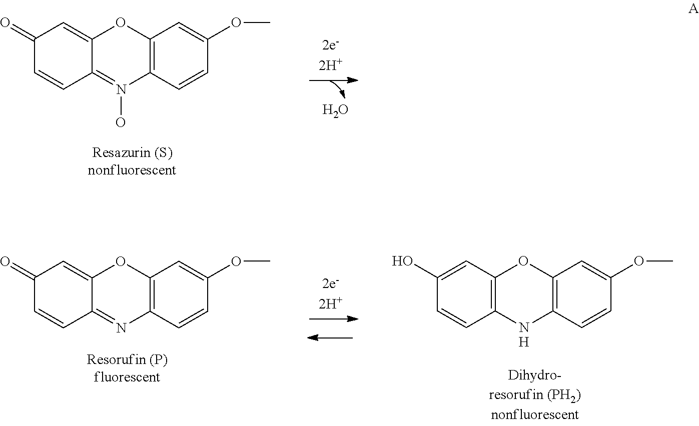

The first reduction step has a midpoint of about +380 mV (depending somewhat on the media) which means that it is easily reduced by all of the usual components of the electron transport chain: FMNH₂, FADH₂, NADH, NADPH and cytochromes. The second step, however, happens at −110 mV that, while not difficult to achieve, will not happen in every case. The presence or absence of a second reduction step can be used to distinguish microbes.

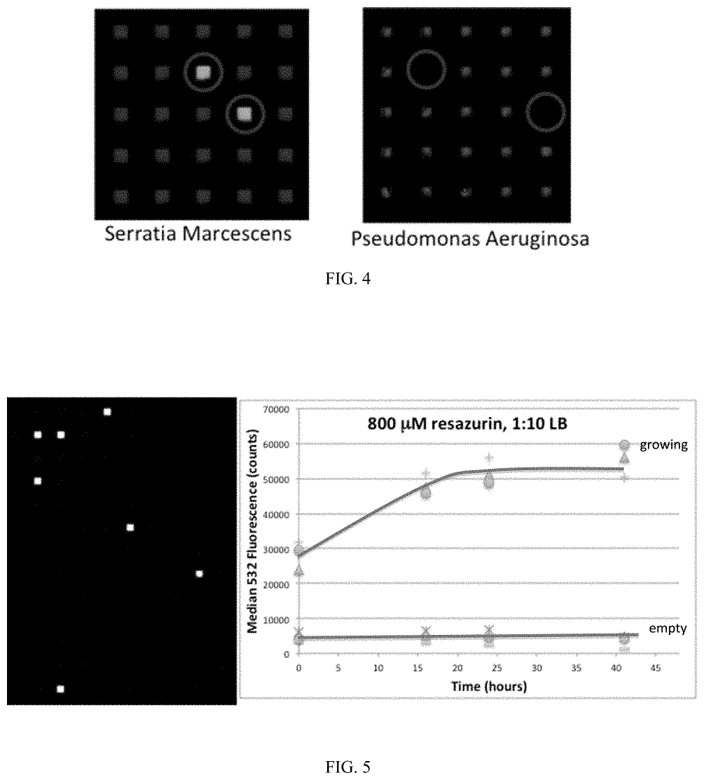

In a test using resazurin as indicator on a microfabricated chip, two different strains, Serratia marcescens and Pseudomonas aeruginosa, both grown in the same R2A media for the same 48 hours produce an obviously different result, as seen in FIG. 4. The wells with serratia show the first reduction step being brighter than the neighboring empty wells. The wells containing pseudomonas are darker because they have gone through both the first and second reduction steps. By choosing some wells that show one reduction step and some wells that show two reduction steps for downstream analysis, diversity of species/isolates transferred from the plurality of microwells can be improved.

Growth dynamics vary strongly according to species. Typically, when placed into fresh media, different strains exhibit a lag phase (slow or no growth), an exponential phase (exponential growth), a stationary phase (no growth), and then death/decline. Different microbes may have long or short lag times or grow to higher or lower stationary states, etc. By following the time-course of the signals of resazurin, a graph of these phases was obtained for each well. See FIG. 5.

Wells with growth are distinct from wells without growth, but even more information can be extracted by monitoring the fluorescence of the wells containing growing cells over time. This microbe has a short lag phase, then grows fast for about one day and plateaus at a high density. Dissimilar time-course signals from different wells can be used to distinguish cell isolates. When many wells display similar time-course profiles in fluorescence, the organisms in these wells may be same or closely related, but further analysis may be needed.

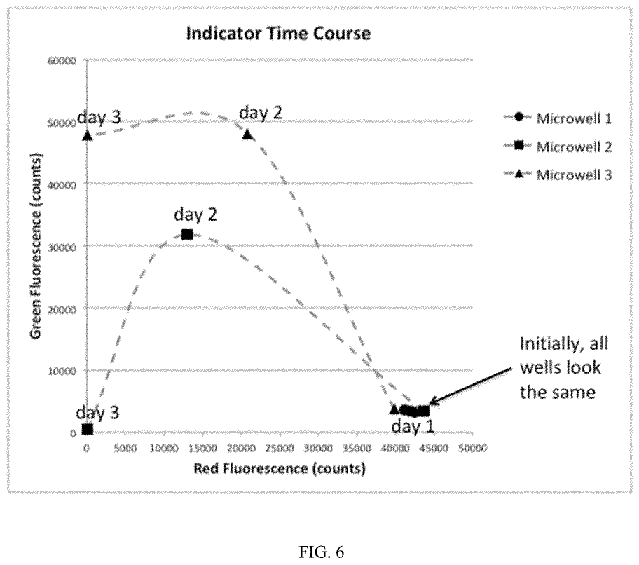

Monitoring resazurin fluorescence with multiple wavelengths at multiple time points provides useful supporting information for selecting a maximally diverse population of microbes to harvest from a chip. In this way, diversity amongst a large number of isolates can be determined before harvesting, which reduces the time/cost of the downstream analysis. In a test, the microwells on a microfabricated chip are either empty, loaded with serratia, or loaded with pseudomonas. Initially all microwells look the same (each well is loaded with resazurin), but data taken on day 2 shows a big difference between wells with bacteria and empty wells, and on day 3, the two bacterial strains are differentiable by the change of the optical property of the indicator. FIG. 6 show time-course behavior of well contents of these microwells on a microfabricated chip with resazurin as indicator, at two fluorescence channels (green/red) over time. In FIG. 6, Microwell 1 indicates those microwells that are empty (no cells loaded or growing); Microwell 2 indicates those microwells that contain pseudomonas, which shows high green on day 2, low green by day 3; Microwell 3 indicates those microwells that contain serratia, which shows high green on day 2 and still high on day 3.

Other parameters, such as change rates of an optical property, intensity of an optical property (which is a subset of change rate at certain time points), the ratio of the signal intensity signals in different wavelength channels can be used to distinguish cell isolates in different wells.

Resazurin is also very sensitive to pH. Microbes that alter the pH of the media they grow in can be studied by watching changes in the resazurin signal. Resorufin becomes increasingly insoluble as the pH drops much below its pKa (around 6.5). The fluorescence signals can be observed regardless of wavelength to drop drastically in intensity at low pH.

While various embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed.

1. A method of using a device comprising a plurality of micro scale experimental units, the method comprising:
in each of the plurality of experimental units, providing at least one cell and an indicator capable of producing an optical signal;
incubating the device at predetermined conditions for a duration of time to grow a plurality of cells from the at least one cell in each of the plurality of experimental units;
measuring at least one optical property of the contents of each of the plurality of experimental units at multiple time points during the incubation, thereby obtaining a time-dependent profile of the optical property for each of the plurality of experimental units; and
analyzing the time-dependent profiles of the at least one optical property for the plurality of experimental units.

2. The method of claim 1, further comprising:
based on the analysis of the time-dependent profiles of the at least one optical property for the plurality of experimental units, determining the presence or absence of a biological entity of interest in at least one of the plurality of experimental units.

3. The method of claim 1, wherein the analysis of the time-dependent profiles of the at least one optical property for the plurality of experimental units comprises a comparison across the time-dependent profiles of the at least one optical property for the plurality of experimental units.

4. The method of claim 3, further comprising:
if the time-dependent profiles of the at least one optical property of two or more of the plurality of experimental units are determined to be sufficiently similar, transferring some of the plurality of cells from only one of the two or more experimental units to a target location, or from a smaller subset of the two or more experimental units each to a respective target location.

5. The method of claim 1, wherein the analysis of the time-dependent profiles of the at least one optical property for the plurality of experimental units comprises identifying one or more features of the time-dependent profiles.

6. The method of claim 5, wherein the one or more features comprises one or more of: the intensity of the optical property at particular time points, the ratio of the intensity of the at least one optical property at different wavelengths, and the rate of change of the at least one optical property with time.

7. The method of claim 1, further comprising:
based on the analysis of the time-dependent profiles of the at least one optical property for the plurality of experimental units, transferring some of the plurality of cells from at least one of the plurality of experimental units to at least one target location.

8. The method of claim 1, further comprising:
if the time-dependent profiles of the at least one optical property of two of the plurality of experimental units are determined to be sufficiently dissimilar, transferring some of the plurality of cells from each of the two experimental units to a respective target location.

9. The method of claim 1, wherein the at least one optical property comprises fluorescence.

10. The method of claim 1, wherein the indicator is capable of emitting fluorescence signals of different wavelength at different redox states.

11. The method of claim 1, wherein the indicator is resazurin.

12. The method of claim 1, wherein the indicator is pH sensitive.

13. The method of claim 2, wherein the at least one biological entity of interest comprises a eukaryotic cell.

14. The method of claim 2, wherein the at least one biological entity of interest comprises bacteria.

15. The method of claim 1, wherein providing the at least one cell comprises loading into each of the plurality of experimental units only one cell.

16. The method of claim 1, wherein the device is a microfabricated device having a top surface defining an array of microwells as experimental units.

17. The method of claim 16, wherein each microwell of the plurality of microwells has a diameter of about 25 μm to about 500 μm.

18. The method of claim 16, and wherein the surface density of the plurality of microwells is at least 750 microwells per cm².

19. A method of using a device comprising a plurality of microscale experimental units, the method comprising:
in each of a plurality of experimental units, providing at least one cell and an indicator capable of producing an optical signal;
incubating the device at predetermined conditions for a duration of time to grow a plurality of cells from the at least one cell in each of the plurality of experimental units;
measuring at least one optical property of the contents of each of the plurality of experimental units during or after the incubation, wherein measuring at least one optical property comprises measuring the at least one optical property of the contents of each of the plurality of experimental units at multiple time points during the incubation;
analyzing the measured at least one optical property for each of the plurality of experimental units, wherein analyzing the measured at least one optical property for each of the plurality of experimental units comprises a comparison among the at least one optical property of each of the plurality of experimental units measured at multiple time points; and
based on the analysis, selecting some of the plurality of cells in one or more of the plurality of experimental units.

20.-23. (canceled)

24. The method of claim 19, wherein analyzing the measured at least one optical property for each of the plurality of experimental units further comprises calculating the ratio of the intensity of the at least one optical property measured at a same time at different wavelengths.

25.-28. (canceled)
